(19) United States
(12) Patent Application Publication
(10) Pub. No.: US 2023/0037749 A1
(43) Pub. Date: Feb. 9, 2023

(54) METHOD AND SYSTEM FOR DETECTING MOOD

(71) Applicant: MatrixCare, Inc., Bloomington, MN (US)

(72) Inventors: Kedar Mangesh KADAM, Bloomington, MN (US); Keegan Duane DSOUZA, Bloomington, MN (US); Christian Michael DROUIN, Bloomington, MN (US); Frank Nash, Bloomington, MN (US)

(21) Appl. No.: 17/785,262

(22) PCT Filed: Nov. 29, 2021
(86) PCT No.: PCT/US2021/061007

§ 371 Date: Jun. 14, 2022

Related U.S. Application Data
(60) Provisional application No. 63/119,505, filed on Nov. 30, 2020.

(51) Int. Cl. G16H 20/70; A61B 5/00; A61B 5/16; G16H 50/20

(52) U.S. Cl. CPC G16H 20/70; A61B 5/7275; A61B 5/165; A61B 5/7267; G16H 50/20

(57) ABSTRACT

A first value for each of a plurality of parameters is received, each of the first values being associated with a user and a first day. A second value for each of the plurality of parameters is received, each of the second values being associated with the user and a second day that is subsequent to the first day. For each of the plurality of parameters, a trend indication is determined, the trend indication being based on the first values, the second values, and a first time period. A base weight value for each of the plurality of parameters is determined, the base weight value being based on the first time period and the determined trend indication associated with the one of the plurality of parameters. A mood score is determined, based on the base weight value for each of the plurality of parameters.

### CROSS-REFERENCE TO RELATED APPLICATIONS

This application claims the benefit of, and priority to, U.S. Provisional Patent Application No. 63/119,505 filed on Nov. 30, 2020, which is hereby incorporated by reference herein in its entirety.

### TECHNICAL FIELD

The present disclosure relates generally to systems and methods for detecting the mood of a user, and more particularly, to systems and methods for detecting long-term changes in mood such as indicating the onset of depression.

### BACKGROUND

Many individuals suffer from mood disorders such as depression to varying degrees. Depression constitutes a leading cause of disability worldwide, due, in part, to its long- and short-term impairment of an individual's motivation, energy, and cognition. In extreme cases, and all too frequently, depression can lead to suicide. Early detection of depression can help in mitigation and improving an individual's quality of life. Unfortunately, there is no quick and economical test for mood disorders such as a blood test. Mood disorders are currently diagnosed by careful examination and observation by health care providers, including nurses, primary care physicians, psychologists, and psychiatrists. Constant monitoring can also be important because, without intervention, a mood trajectory can progressively and unexpectedly lead to depression. Coupled with barriers including perceived or real social stigma, treatment cost, and treatment availability, some individuals are not diagnosed early enough, leading to deterioration in their condition and quality of life, and exacerbating the costs and treatment challenges. The present disclosure is directed to solving these and other problems.

### SUMMARY

According to some implementations of the present disclosure, a method includes receiving a first value for each of a plurality of parameters, each of the first values being associated with a user and a first day. The method further includes receiving a second value for each of the plurality of parameters, each of the second values being associated with the user, and a second day that is subsequent to the first day. The method further includes determining, for each of the plurality of parameters, a trend indication, the trend indication for each of the plurality of parameters being based at least in part on the first values, the second values, and a first time period. The method further includes determining a base weight value for each of the plurality of parameters, the base weight value for each one of the plurality of parameters being based at least in part on the first time period, and the determined trend indication associated with the one of the plurality of parameters. The method further includes determining a mood score based on the base weight value for each of the plurality of parameters.

According to some implementations of the present disclosure, a system includes (i) a control system including one or more processors and (ii) a memory having stored thereon machine readable instructions. The control system is coupled to the memory. Any of the methods disclosed herein is implemented when the machine executable instructions in the memory are executed by at least one of the one or more processors of the control system.

According to some implementations of the present disclosure, a system for determining a mood score includes a control system configured to implement any of the methods disclosed herein.

According to some implementations of the present disclosure, a computer program product comprising instructions which, when executed by a computer, cause the computer to carry out any of the methods disclosed herein.

According to some implementations of the present disclosure, a system for diagnosing a user based on a mood score includes one or more sensors, a memory, and a control system. The one or more sensors are configured to generate a plurality of parameters associated with the user. The memory stores machine-readable instructions. The control system includes one or more processors configured to execute the machine-readable instructions to receive a first value for each of the plurality of parameters. Each of the first values is associated with (i) the user and (ii) a first day. The control system is further configured to receive a second value for each of the plurality of parameters. Each of the second values is associated with (i) the user and (ii) a second day that is subsequent to the first day. The control system is further configured to determine, for each of the plurality of parameters, a trend indication. The trend indication for each of the plurality of parameters is based at least in part on the first values, the second values, and a first time period. The control system is further configured to determine a base weight value for each of the plurality of parameters. The base weight value for each of the plurality of parameters is based at least in part on the first time period and the associated determined trend indication. The control system is further configured to determine the mood score, based on the base weight value for each of the plurality of parameters.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 is a perspective view of at least a portion of the system of FIG. 1 and a user, according to other implementations of the present disclosure;

FIG. 4 is a perspective view of at least a portion of the system of FIG. 1 and a user, according to yet other implementations of the present disclosure;

FIG. 5 is a first plot of user parameters according to some implementations of the present disclosure;

FIG. 6 is a second plot of user parameters according to some implementations of the present disclosure;

FIG. 7 is a third plot of user parameters according to some implementations of the present disclosure; and

FIG. 8 is a flowchart depicting a process for determining a mood score according to some aspects of the present disclosure;

FIG. 9 is a flowchart depicting steps for a machine learning algorithm;

FIG. 10 is a plot depicting sensor data;

FIG. 11 is a plot depicting point of care (POC) data;

FIG. 12 is a plot depicting progress notes data; and

FIG. 13 is a plot depicting control data.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Where a person's mood can affect their quality of life, long-term trends are often more important in mitigating and avoiding more serious issues such as chronic anxiety, mania, and depression. For example, sadness is a normal reaction to loss, disappointment, or other difficulties, and will typically go away with time. In contrast, depression is a mood disorder that can manifest as sadness but is often persistent and can come up for no reason. Along with sadness, anger, and irritability, depressed individuals sometimes have feelings of worthlessness, hopelessness, unreasonable guilt. The individuals themselves may not understand their mood or mental state, and when they do, either by self-reflection or by a medical or care provider's diagnosis, an individual or the medical or care provider can work to redirect and adjust their mood. Monitoring a person's mood and understanding if an individual is sliding into, or suddenly transitioning into, an undesirable mental state such as depression, is important for self-mitigation and intervention.

Humans are hardwired to detect a person's mood. Beyond explicit verbal communication, the nuances in a person's speech and their physical demeanor, such as facial expressions, provide clues that humans categorize and, often subconsciously, allow others to understand a person's mental state. Similarly, humans, albeit in some cases only specialized humans with appropriate medical training, can determine mood disorders such as depression. Considering the somewhat intuitive nature of determining mood and mood disorders, detection of mood and mood disorders by analytical tests or sensors is a surprising, more significant challenge. However, as described herein, bringing to bear multiple sensors and the power of modern computing, mood and mood disorders can be detected.

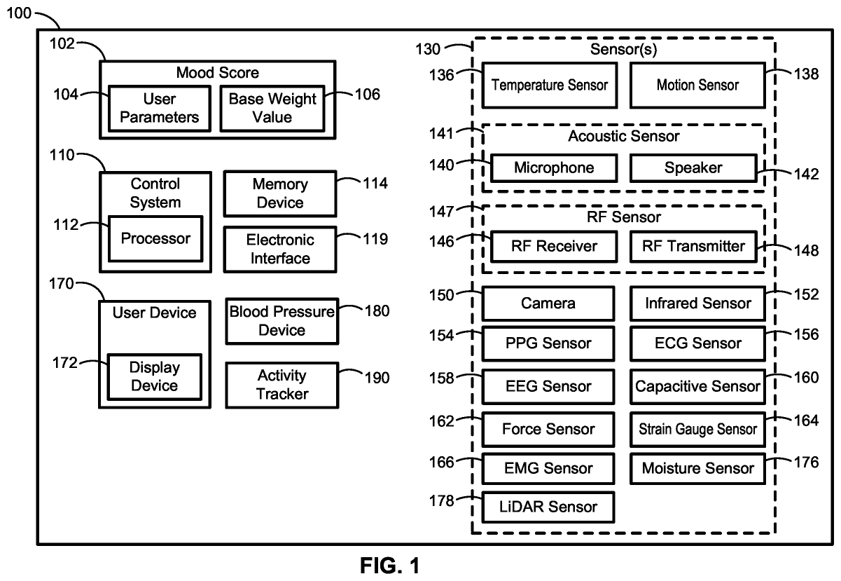

Referring to FIG. 1, a system 100, according to some implementations of the present disclosure for determining a mood of a user for diagnosis and treatment of mental diseases and conditions, is illustrated. The system 100 includes a mood score module 102, a control system 110, a memory device 114, an electronic interface 119, one or more sensors 130, and one or more user devices 170. As described in more detail herein, the user device 170 also includes a display device 172. In some implementations, the user device 170 includes physical interface(s) to the one or more sensors 130. In some implementations, the system 100 further optionally includes a blood pressure device 180, an activity tracker 190, or any combination thereof.

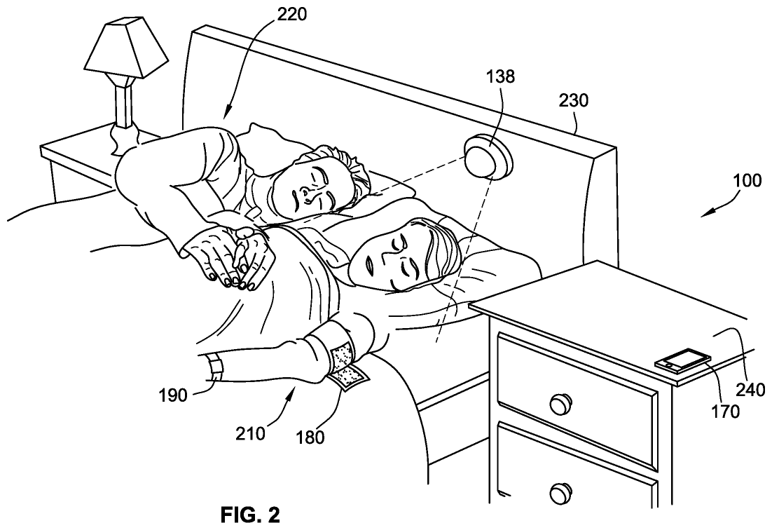

The mood score module 102 determines a mood score for a user based at least on parameters 104 (e.g., user parameters) and base weight values 106. The mood score is indicative of the mood of a user. The user parameters 104 include data that are collected by the one or more sensors 130, examples of which are shown in FIGS. 2-4 herein. The base weight values 106 are modifiers applied to the parameters depending on the importance of a specific parameter. That is, the mood score (or mood score module) 102 is a function of both the user parameters 104 and the base weight values 106.

In some implementations, such as responsive to a determined mood score or a mood score range, the system 100 can be used to diagnose, treat and/or recommend for treatment a variety of mood disorders or psychological disorders. In some such implementations, the system 100 can diagnose, treat, and/or recommend for treatment major depression, dysthymia, bipolar disorder, substance-induced mood disorder, mood disorder related to another health condition, or any combination thereof. Additionally or alternatively, in some implementations, the system 100 can diagnose, treat, and/or recommend for treatment major depressive disorder, bipolar disorder, seasonal affective disorder, cyclothymic disorder, premenstrual dysphoric disorder, persistent depressive disorder (or dysthymia), disruptive mood dysregulation disorder, depression related to medical illness, depression induced by substance use or medication, or any combination thereof. Additionally or alternatively, in some implementations, the system 100 can diagnose, treat, and/or recommend for treatment ADHD, anxiety, social phobia, etc. The treatment and/or recommended treatment can include medications (e.g., antidepressants, stimulants, mood-stabilizing medicines), psychotherapy, family therapy, other therapies, or any combination thereof. For example, in some implementations, the system 100 provides automatic treatment of the patient, such as automatic generation of prescription of the medication(s), and/or automatic administration of the prescribed medication(s).

The mood score can be any useful representation or value, such as a number, a word, a string of text, a letter, a symbol, or a string of machine-readable code. In some implementations, a mental state of the user is determined using system 100 based at least in part on the determined mood score. Without limitation, as used herein, the mental state includes one or more of mania, happiness, euthymia or a neutral mood, sadness, depression, anxiety, apathy, and irritability. For example, the mental state is determined to be a first mental state responsive to the mood score satisfying a first range of values, and the mental state is determined to be second mental state responsive to the mood score satisfying a second range of values. It is recognized that mental states present a spectrum of overlapping states, such as when the first range of values and the second range of values have a range of overlapping values. In this case, the mental state is determined to include the first mental state and the second mental state.

In some implementations, the mental state is determined, responsive to a plurality of mood score range values, to be one or more of: (i) mania responsive to the mood score satisfying a first range of mood score values; (ii) happiness responsive to the mood score satisfying a second range of mood score values; (iii) euthymia or a neutral mood responsive to the mood score satisfying a third range of mood score values; (iv) sadness responsive to the mood score satisfying a fourth range of mood score values; (v) depression responsive to the mood score satisfying a fifth range of mood score values; (vi) anxiety response to the mood score satisfying a sixth range of mood score values; (vii) apathy responsive to the mood score satisfying a seventh range of mood score values; and (vii) irritability responsive to the mood score satisfying an eighth range of mood score values. As previously described, any one or more of the plurality of mood score ranges can overlap with one or more of a different mood score range, indicative of overlapping mental states.

In some implementations, a representation of the mood score or of the mental state is communicated to the user or a care provider. In some implementations, the mood score is automatically classified for a diagnosis of one of the mental conditions, such as mood disorders described here. In some such implementations, the diagnosis includes depression, dysthymia, bipolar disorder, substance-induced mood disorder, mood disorder related to another health condition, or any combination thereof. Additionally or alternatively, in some implementations, the diagnosis includes major depressive disorder, bipolar disorder, seasonal affective disorder, cyclothymic disorder, premenstrual dysphoric disorder, persistent depressive disorder (or dysthymia), disruptive mood dysregulation disorder, depression related to medical illness, depression induced by substance use or medication, or any combination thereof. The representation of the mood score or mental state can be communicated by any means, such as on a display device 172 of the user device 170. In some implementations, the display provides a graphical representation, e.g., a pictogram of a happy face, neutral face, a sad face, etc.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special-purpose processor or microprocessor. While one processor 112 is shown in FIG. 1, the control system 110 can include any suitable number of processors (e.g., one processor, two processors, five processors, ten processors, etc.) that can be in a single housing or located remotely from each other. The control system 110 can be coupled to and/or positioned within, for example, a housing of the user device 170, the activity tracker 190, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer-readable storage device or media, such as, for example, a random or serial access memory device, a hard drive, a solid-state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned, within a housing of the user device 170, the activity tracker 190, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user, which can be implemented as user parameters 104 for determination of the mood score 102. The user profile can include, for example, demographic information associated with the user, biometric information associated with the user, medical information associated with the user, self-reported user feedback, sleep parameters associated with the user (e.g., sleep-related parameters recorded from one or more sleep sessions), or any combination thereof. The demographic information can include, for example, information indicative of an age of the user, a gender of the user, a race of the user, a family history of mental health, an employment status of the user, an educational status of the user, a socioeconomic status of the user, or any combination thereof. The medical information can include, for example, including indicative of one or more medical conditions associated with the user, medication usage by the user, or both. The self-reported user feedback can include information indicative of a self-reported subjective mood and mental health, a self-reported subjective stress level of the user, a self-reported subjective fatigue level of the user, a self-reported subjective health status of the user, a recent life event experienced by the user, or any combination thereof. The user profile information can be updated at any time, such as daily, weekly, monthly, or yearly.

In some implementations, the user profile can include clinical and or therapy session notes and assessments. For example, an assessment from any one or more of a care provider, nurse, and medical professional. These can include data from the electronic health record (EHR), a minimal data set (MDS), and point of care data (POC). The user profile can include a mood assessment based on a patient health questionnaire, for example, a PHQ9. The assessment can also include other observations such as visual changes in appearance, weight changes, energy level changes, mannerism changes, and changes in medication. The user profile can also include information regarding deaths of the user's loved ones, such as a spouse, companion, or pet.

The electronic interface 119 is configured to receive data from the one or more sensors 130 such that the data can be stored in the memory device 114 and/or analyzed by the processor 112 of the control system 110. The received data, such as physiological data and/or audio data, is included as user parameters 104 for determination of the mood score 102. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one or more processors and/or one or more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The one or more sensors 130 of the system 100 include a temperature sensor 136, a motion sensor 138, a microphone 140, a speaker 142, a radio-frequency (RF) receiver 146, an RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the moisture sensor 176, and the LiDAR sensor 178, more generally, the one or more sensors 130 can include any combination and any number of each of the sensors described and/or shown herein.

FIG. 2 is an illustration of an environment 200 according to some implementations where a portion of the system 100 (FIG. 1) is used. A user 210 of the system 100, and a bed partner 220 are located in a bed 230 and are laying on a mattress 232. A motion sensor 138, a blood pressure device 180, and an activity tracker 190 are shown, although any one or more sensors 130 can be used to generate or monitor user parameters 104 during a sleeping or resting session of user 210.

In some implementations, physiological data generated by one or more of the sensors 130 can be used by the control system 110 to determine the duration of sleep and sleep quality of user 210, which is a user parameter 104. For example, a sleep-wake signal associated with the user 210 during a sleep session and one or more sleep-related parameters can be determined. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

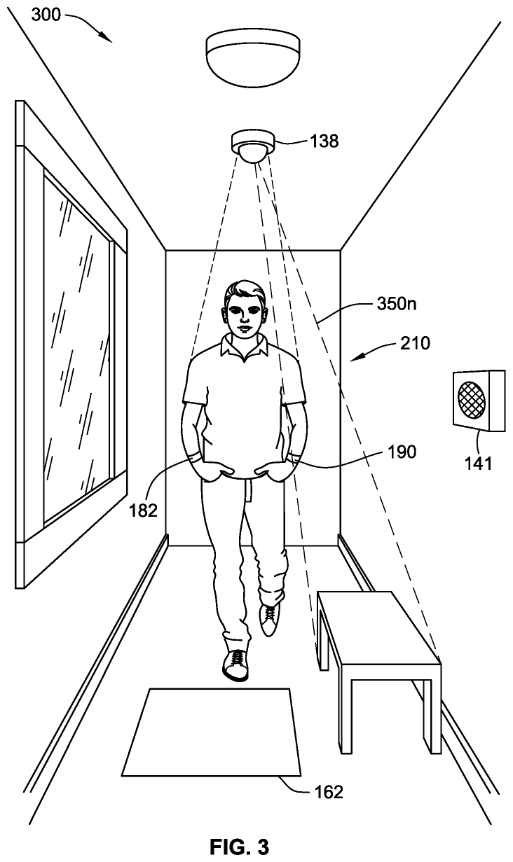

FIG. 3 illustrates another environment 300 according to some implementations where a portion of system 100 (FIG. 1) is used. The user 210 is shown walking down a hallway. A motion sensor 138, a force sensor 162, an acoustic sensor 141, and an activity tracker 190 are also shown. The environment 300 can be a resident's home (e.g., house, apartment, etc.), an assisted living facility, a hospital, etc. Other environments are contemplated. As shown, a motion sensor 138 is configured to detect via transmitted signals 351n a position of the resident (e.g., the user 210). Any one or more of the sensors 130 can be used to monitor user 210 and generate user parameters 104, such as activity data, audio data, or both.

In some implementations, physiological data generated by one or more of the sensors 130 can be used by the control system 110 to determine user parameters 104, in environment 300, and the like. Specifically, in environment 300, the physical activity and movement of user 210 can be determined. For example, the sensor 138 is configured to generate data (e.g., location data, position data, physiological data, etc.) that can be used by the control system 110 to determine user parameters 104 of the user 210.

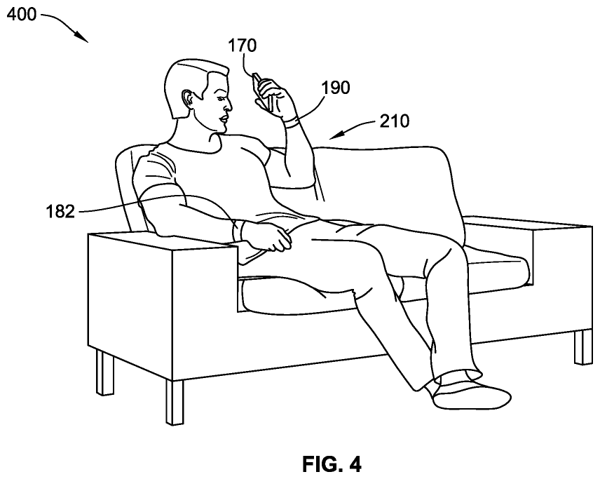

FIG. 4 illustrates yet another environment 400 according to some implementations where a portion of system 100 (FIG. 1) is used. The user 210 is shown sitting and speaking into a user device 170. A motion sensor 138 and an activity tracker 190 are also shown. Any one or more of the sensors 130 can also be used in this environment.

Physiological data and audio data generated by one or more of the sensors 130 can be used by the control system 110 to determine one or more user parameters 104 associated with user 210. For example, the user device 170 can include a Chatbot application to ask questions and monitor replies from the user. The replies provide user parameters 104 to determine a mood score.

In some implementations, a Chatbot application, such as implemented using user device 170 or acoustic sensor 141, detects one or more of a plurality of parameters 104 including a spoken language during verbal communication, content of language during verbal communication, speed of talking during verbal communication, length of pauses between sentences during verbal communication, mean pitch during verbal communication, peak pitch during verbal communication, mean volume during verbal communication, peak volume during verbal communication, minimal volume during verbal communication, force on a keyboard during typed communication, speed of typing during typed communication, length of pauses between entries during typed communication, frequency of communication during typed communication, frequency of communication during verbal communication, and confidence of user speech during verbal communication. In addition to these verbal communications, one or more of breathing rate information, heart rate information, temperature information, physical activity information, blood pressure information, social media interaction information, mood information, interest or pleasure in activities, facial expression information, tiredness, and overall energy, can also be determined using one or more of the sensors 130, heart rate tracker 182, and activity tracker 190.

In addition to how the user speaks, the Chatbot can capture the content of the user's speech. The Chatbot can pose standard questions, such as could be posed by a care provider. For example, “how are you feeling,” “what did you eat today,” “did you sleep well,” “did you take your medication,” “what are your plans for today” etc. Optionally, the Chatbot can be used by a care provider to communicate user-relevant data, such as vitals and answers to the standard questions.

FIGS. 2 to 4 illustrate some environments where the system 100 or a portion of system 100 can be implemented. Other environments are also conceived, such as the outdoors, public spaces, private homes, in a car, etc. For example, an activity tracker 190 and a user device 170 such as a smartphone can be portable/wearable and implemented in most environments.

Returning to FIG. 1, the temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, an ambient temperature, or any combination thereof. The temperature sensor 136 can be, for example, a thermocouple sensor, a thermistor sensor, a silicon bandgap temperature sensor or semiconductor-based sensor, a resistance temperature detector, or any combination thereof.

The microphone 140 outputs audio data that can be stored in the memory device 114 and/or analyzed by the processor 112 of the control system 110. The audio data generated by the microphone 140 is reproducible as one or more sound(s), e.g., sounds from the user 210, during a sleep session (FIG. 2), during active movement (FIG. 3), or can be a part of user device 170 (FIG. 4). The audio data from the microphone 140 can also be used to identify (e.g., using the control system 110) an event experienced by the user during sleep, activity, or when interacting with a user device 170. The microphone 140 can be coupled to or integrated in the user device 170 or in acoustic sensor 141.

The speaker 142 outputs sound waves that are audible to a user of the system 100 (e.g., the user 210 of FIGS. 2 to 4). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to the user device 170 or integrated with acoustic sensor 141.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval, and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 and/or one or more of the sleep-related parameters described herein.

In some implementations, the sensors 130 include (i) a first microphone that is the same as, or similar to, the microphone 140 and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high-frequency band, within a low-frequency band, longwave signals, short wave signals, etc.). The RF receiver 146 detects the reflections of the radio waves emitted from the RF transmitter 148, and this data can be analyzed by the control system 110 to determine a location of the user 210 (e.g., FIGS. 2 to 4) and/or one or more of the user parameters 104 described herein. An RF receiver (either the RF receiver 146 and the RF transmitter 148 or another RF pair) can also be used for wireless communication between the control system 110, the one or more sensors 130, the user device 170, the blood pressure device 180, the activity tracker 190, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which includes an RF sensor that is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving and partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the user parameters 104 described herein. For example, the image data from the camera 150 can be used to identify a location of the user, to determine a time when the user 210 enters a bed 230 (FIG. 2), and to determine a time when the user 210 exits the bed 230.

In some implementations, the camera can be used to identify the user 210 by facial features. The camera 150 can also be used to identify changes in the user's facial features. For example, facial features indicative of mood can be monitored by camera 150. In some implementations, a facial tracking and mood detecting application is used, such as concurrently with or as a part of a Chatbot.

The infrared (IR) sensor 152 outputs infrared image data reproducible as one or more infrared images (e.g., still images, video images, or both) that can be stored in the memory device 114. The infrared data from the IR sensor 152 can be used to determine one or more user parameters 104 during a sleep session (FIG. 2), during daily activities (FIG. 3), or when user 210 is interacting with user device 170. The user IR sensor can detect a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (e.g., FIGS. 2 to 4) that can be used to determine one or more user parameters 104, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, such as implemented as part of user device 170 or another wearable device, or embedded in clothing and/or fabric that is worn by the user 210.

The ECG sensor 156 outputs physiological data associated with the electrical activity of the heart of the user 210. In some implementations, the ECG sensor 156 includes one or more electrodes that are positioned on or around a portion of the user 210 during the sleep session (FIG. 2). Alternatively, a wearable ECG sensor can be applied to user 210, such as on their chest, while they are active and out of bed (e.g., FIG. 3 or 4). The physiological data from the ECG sensor 156 can be used, for example, to determine one or more of the sleep-related parameters described herein.

The EEG sensor 158 outputs physiological data associated with the electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in user wearable devices, such as a headband or hat, and used when the user is out of bed (e.g., FIGS. 3 and 4).

The capacitive sensor 160, the force sensor 162, and the strain gauge sensor 164 output data that can be stored in the memory device 114 and used by the control system 110 to determine one or more of the user parameters 104 described herein. The EMG sensor 166 outputs physiological data associated with electrical activity produced by one or more muscles. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The moisture sensor 176 outputs data that can be stored in the memory device 114 and used by the control system 110. The moisture sensor 176 can be used to detect moisture in various areas surrounding the user. The moisture sensor 176 is placed near any area where moisture levels need to be monitored. The moisture sensor 176 can also be used to monitor the humidity of the ambient environment surrounding the user 210, for example, the air inside a bedroom (FIG. 2) or another user environment (e.g., FIG. 3).

The Light Detection and Ranging (LiDAR) sensor 178 can be used for depth sensing. This type of optical sensor (e.g., laser sensor) can be used to detect objects and build three-dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different types of obstacles.

While shown separately in FIG. 1, any combination of the one or more sensors 130 can be integrated in and/or coupled to any one or more of the components of the system 100, including, the control system 110, the user device 170, or any combination thereof. For example, the microphone 140 and speaker 142 are integrated in and/or coupled to the user device 170. In some implementations, at least one of the one or more sensors 130 is not coupled to, the control system 110, or the user device 170, and is positioned generally adjacent to the user 210 during the sleep session (FIG. 2) or during various activities (e.g., FIG. 3 or 4).

The user device 170 (FIG. 1) includes a display device 172. The user device 170 can be, for example, a mobile device such as a smartphone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smartwatch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 180 is generally used to aid in generating physiological data for determining one or more blood pressure measurement user parameters 104. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by a user and a pressure sensor. For example, as shown in the example of FIG. 2, the blood pressure device 180 can be worn on an upper arm of the user 210. In such implementations where the blood pressure device 180 is a sphygmomanometer, the blood pressure device 180 also includes a pump (e.g., a manually operated bulb) for inflating the cuff. More generally, the blood pressure device 180 can be communicatively coupled with, and/or physically integrated in (e.g., within a housing), the control system 110, the memory 114, the user device 170, and/or the activity tracker 190.

The activity tracker 190 is generally used to aid in generating physiological data for determining activity measurement-related user parameters. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIGS. 2 to 4, the activity tracker 190 is worn on a wrist of the user 210. The activity tracker 190 can also be coupled to or integrated a garment or clothing that is worn by the user. Alternatively, the activity tracker 190 can also be coupled to or integrated in (e.g., within the same housing) the user device 170. More generally, the activity tracker 190 can be communicatively coupled with, or physically integrated in (e.g., within a housing), the control system 110, the memory 114, the user device 170, and/or the blood pressure device 180.

While the control system 110 and the memory device 114 are described and shown in FIG. 1 as being a separate and distinct component of the system 100, in some implementations, the control system 110 and/or the memory device 114 are integrated in the user device 170. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for generating user parameter data 104 and determining a mood score 102 of the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As a further example, a fourth alternative system includes the control system 110, the memory device 114, at least one of the one or more sensors 130, the user device 170, and the blood pressure device 180 and/or activity tracker 190. Thus, various systems can be formed using any portion or portions of the components shown and described herein and/or in combination with one or more other components.

As previously described with reference to FIG. 1, the mood score 102 is a function of the user parameters 104 and base weights 106. An implementation of base weights is shown with reference to Table 1, which lists base weight values and healthy thresholds for steps taken by a user.

TABLE 1

| Exemplification | | | | | | | | | | |
|---|---|---|---|---|---|---|---|---|---|---|
| of Base | | | | | | | | | | |
| Weights | Healthy | | Short-term time | | | | Long-term time | | | |
| User | Thresholds | | | Stable- | Stable- | | | Stable- | Stable- | |
| Parameter | Low | High | + | good | bad | − | + | good | bad | − |
| Steps | 1500 | 3000 | 1 | 5 | 15 | 6 | 7 | 11 | 21 | 12 |

A parameter value is considered healthy if it is between the low and the high thresholds. In this implementation, if the user is taking between 1500 and 3000 steps, these are considered healthy values. Below 1500 steps, the user may be considered too sedentary, and this can be an indication of a mood change, for example, if a user typically will take more than 1500 steps per day. The user exceeding 3000 steps can also be an indication of a mood change, for example, mania, anxiety, or frustration. The healthy thresholds can be initially set based on the user profile associated with the user (e.g., demographic information, medical information, age, and gender). In some implementations, there is only a low threshold or only a high threshold. The initially set thresholds can be adjusted based on changes in the user profile. For example, where a user starts being much more active due to a lifestyle change, the healthy threshold for the example of Table 1 can be increased above 3000 steps. Conversely, a user who may have a new health issue, such as a broken hip due to a fall, would require a downward shift in the low and high thresholds for steps taken.

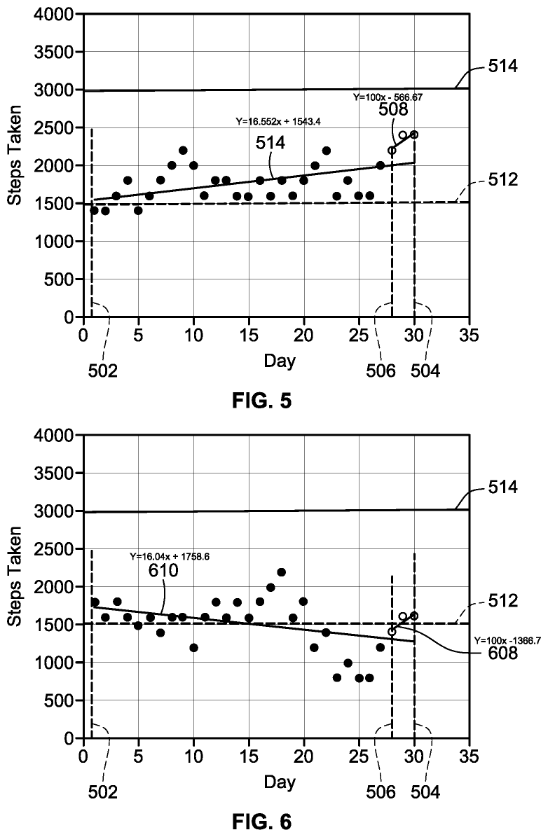

Base weights for a short-term time and a long-term time are listed in Table 1. These are further categorized responsive to the trends seen for the user parameters over time. A “+” indicates a positive or good trend, “−” indicates a negative or bad trend. The category “stable-good” denotes that the trend is stable and within the healthy thresholds. The category “stable-bad” denotes that the trend is stable, but the parameter or some combination of the parameters is outside the healthy thresholds. These aspects will be described in more detail with reference to the following description and FIGS. 5 to 7.

An implementation for the selection of base weights is further illustrated by FIG. 5. A graph is shown for the parameter of user steps taken. For each day, a total number of steps is recorded, for example, using an activity tracker 190 (FIGS. 1 and 2). The current day, day 30, is the last recorded day. A long time period (or a first time period) is selected to include a first day 502, day 1, to a second day 504, day 30. A short time period (or an intermediate time period) is selected to include an intermediate day 506, day 28, to the second day 504. The trend for the short time period and long time period is then categorized as positive (“+”) or negative (“−”) for the determination of the base weight. The designation positive (“+”) or negative (“−”) is a trend indicator. The determination of the trend can be done by any useful means, such as by linear regression. In this implementation, the short-term trend 508 and the long-term trend 510 are determined by linear regression to provide corresponding slopes. The slope of line 508 is 100, and the slope of line 510 is 17. The low healthy threshold 512 and high healthy threshold 514 are indicated. An increase of steps taken is considered a positive trend indicator, and since both line 508 and line 510 have positive slopes, the trend indication is categorized as positive, “+.” Accordingly, for the data plotted in FIG. 5, the base weight for the short-term time is determined from Table 1 to be 1, and the base weight for the long-term time is determined from Table 1 to be 7.

A positive or good trend indication generally indicates that the values associated with a parameter during a given time period are trending in a desired direction. Conversely, a negative trend indication generally indicates that the values associated with a parameter during a given time period are trending away from the desired direction. For user steps taken, the trend indication takes the sign of the slope. For example, a positive slope indicates a positive trend indicator, and a negative slope denotes a negative trend indicator. For some other user parameters, the trend indication may take the opposite sign of the slope. For example, if the user parameter is blood pressure, the relationship is reversed. That is, generally, a lowering of blood pressure would denote a positive trend indicator, and an increase in blood pressure would denote a negative trend indicator.

FIG. 6 shows an alternative set of data for the user where different based weights are determined. The slope of the long-term fitted line 610 is −16. The slope of the short-term fitted line 608 is 100. Accordingly, the long-term trend indication is negative (“−”), and the short-term trend indication is positive (“+”). Therefore, for the data plotted in FIG. 6, the base weight for the short-term time is determined from Table 1 to be 1, and the base weight for the long-term time is determined from Table 1 to be 12.

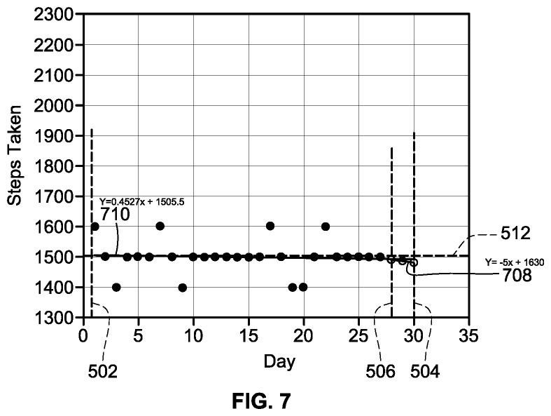

FIG. 7 shows yet another data set of steps taken by the user. The slope of the long-term fitted line 710 is −0.4. The slope of the short-term fitted line 708 is −5. The short trend indicator is therefore negative (“−”), and the short-term base weight is determined to be 6 from Table 1. Although the fitted line 710 has a negative slope, the slope magnitude is small, and the parameter can be classified as “stable.” To quantify the stability of a parameter, the trend can be further categorized by a threshold. For example, in this implementation, where the magnitude of the slope of the fitted data is less than 0.5, the trend is classified as stable. Using this threshold, the long-term time trend indicator is classified as “stable.” In addition to being stable, the steps taken are very close to the lower health threshold limit. The last data point, the average of the last three data points, and the average of all the data points are below the 512 threshold of 1500 steps. In some implementations, where the current day parameter (second day 504), or an average of the parameters in the time period considered, is outside of a healthy parameter, the trend is categorized as “bad.” Therefore, the long-term trend indicator for the data illustrated in FIG. 7 is determined to be “stable-bad.” The long-term base weight selected from Table 1 is accordingly 21

While the range of slope threshold values has been described above as being between about −0.5 and about 0.5, more generally, the upper and lower slope threshold values can be any suitable number (e.g., between about −0.05 and about 0.05, between −0.01 and about 0.01, between about −0.1 and about 0.1, between about −0.3 and about 0.3, between about −0.4 and about 0.4, etc.).

Other user parameters can be treated similarly as described for user steps taken. In some implementations, a plurality of data points associated with user parameters, such as provided by one or more of sensors 130, can be used to determine a trend indication for each of the user parameters. In some implementations, the data set for each of the user parameters is normalized. This normalization can simplify the analysis and manipulations, for example, by allowing selection of a meaningful and single upper and single lower slope thresholds, and having base weight values of similar magnitude for all the parameters.

Other categories of additional base weights and for determining the additional base weights are contemplated. For example, where a trend is positive but entirely outside of the healthy thresholds, a category of “positive-bad” can be used. For a trend that is negative and entirely outside the healthy thresholds, a category of “negative-bad” can be used.

Although only describing a long time period and a short time period, other additional time periods are contemplated. For example, a very long time period can be greater than the long time period. Although the total specified days for the long-term time included days 1 through 30, longer periods of time can be used. For example, user parameters can be collected for more than 30 days, more than three months, more than six months, more than a year, or for more than several years (e.g., 2, 3, 4, 5, or more years).

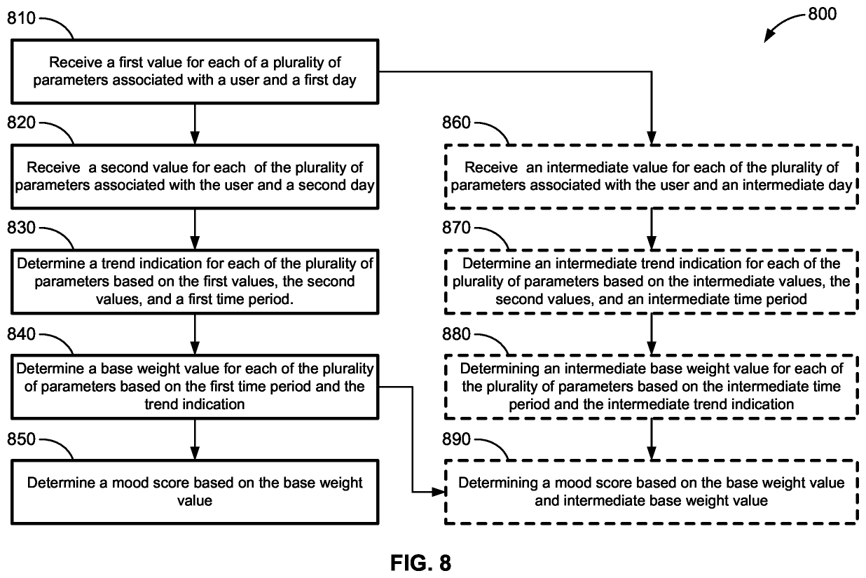

FIG. 8 is a flowchart depicting a process 800 for implementation of module 102 (FIG. 1). The process 800 is for determining a mood score, according to certain aspects of the present disclosure. Process 800 can be performed by any suitable computing device(s), such as any device(s) of system 100 of FIG. 1. In some implementations, process 800 can be performed by a smartphone, tablet, home computer, or other such devices.

The process 800 includes receiving values for a plurality of parameters, which are the user parameters 104 (FIG. 1) associated with a user in need of determining a mood score. In block 810, a first value for each of the plurality of parameters associated with the user is received on a first day. In block 820, a second value for each of the plurality of parameters associated with the user is received on a second day.

The parameters are as previously described including user profile information that can be stored on memory device 114 and physiological data provided by sensors 130 (FIG. 1). In some implementations, each of the plurality of parameters is determined based at least in part on data generated by one or more sensors 130. Optionally the one or more sensors 130 is a microphone 140, camera 150, a pressure sensor (e.g., part of a blood pressure monitoring device 180) a temperature sensor 136, or a motion sensor 138. In some implementations, at least one sensor is physically coupled to or integrated with a user device 170. Optionally, at least one sensor is physically coupled to an activity tracker 190. In some cases, at least one sensor is physically coupled to a heartrate monitor.

As previously described, in some implementations, the plurality parameters include verbal communication, such as the user verbal communication and interaction with a Chatbot. Optionally, the parameter includes the percentage of non-primary language spoken by the user. The parameter can optionally include the number of swear or frustration words spoken by the user. The parameter can also optionally include the mean volume during verbal communication is an average of the measure of volume in decibel (dB) of spoken words. Optionally, the peak volume during verbal communication is a user parameter wherein the highest measured volume in dB within 2 standard deviations of the mean volume is measured. In some implementations, the minimal volume during verbal communication is a user parameter and the lowest volume in dB within 2 standard deviations of the mean volume is measured.

In some implementations, the user's facial expression is a user parameter. The number of times a particular expression occurs can be measured. For example, the number of times a person smiles. Alternatively, the number of times a user frowns.

In some implementations where blood pressure information is a user parameter, a systolic component and a diastolic component can be independent or combined parameters. In some implementations, the user parameter is the heart rate information, wherein the average beats per minute are measured.

According to some aspects, a social media interaction is a user parameter. The social media interaction can be using the user device 170 or any other device. The number of times and/or time spent on social media can be monitored. The content accessed can be monitored. Social media interaction can also be included with the Chatbot application.

The user parameters received in steps 810, 820 are used to determine a trend indication in block 830 for each of the user parameters. The trend indication is based at least on the first values, the second values, and a first time period. The first time period is the period of time between the first day and the second day. The trend indication can be determined by statistical methods such as line fitting as previously described with reference to FIGS. 5 to 7.

In some implementations, the trend indication for each of the plurality of parameters is also based at least in part on a range of healthy threshold values for each of the plurality of parameters. Optionally, determining the trend indication for each of the plurality of parameters includes determining a rate of change between at least the first value and the second value for each of the plurality of parameters during the first time period. For example, the rate of change is associated with a slope of a line that is fitted to at least the first value and the second value.

In some implementations, the trend indication for a first one of the plurality of parameters is a positive trend indication responsive to a determination that the slope of the fitted line is greater than a first slope threshold. For example, wherein the first slope threshold is 0.5, 0.3, 0.2, 0.1, 0.01, or 0.05.

In some implementations, the trend indication for a first one of the plurality of parameters is a negative trend indication responsive to a determination that the slope of the fitted line is less than a second slope threshold. For example, wherein the second slope threshold is −0.5, −0.3, −0.2, −0.1, −0.01, or −0.05.

Optionally, the trend indication for a first one of the plurality of parameters is a stable-good trend indication responsive to determining that; (i) the slope of the fitted line is within a range of slope values, and (ii) an average of at least the first value and the second value is within a range of healthy threshold values. In some implementations, at least one of the first value and the second value are within the range of healthy threshold values for the trend indication to stable-good. In some implementations, the first value and the second value are within the range of the healthy threshold values for the trend indication to be stable-good.

Optionally, the trend indication for a first one of the plurality of parameters is a stable-bad trend indication responsive to determining that; (i) the slope of the fitted line is within a range of slope values, and (ii) an average of at least the first value and the second value is outside a range of healthy threshold values. In some implementations, at least one of the first value and the second value are outside of the range of healthy threshold values for the trend indication to be stable-bad. In some implementations, at least the first value and the second value are outside of the range of healthy threshold values for the trend indication to be stable-bad.

A base weight value for each of the plurality of parameters is determined in block 840. The base weight value is determined based on the first time period and the associated trend indication. The based weigh value can be determined as previously described, e.g., Table 1, FIGS. 5 to 7, using the trend indication and first time period as criteria. For example, in the data presented in Table 1, the first time period can refer to the long-term time.

In block 850 the mood score 120 (FIG. 1) is determined based at least on the base weight value for each of the plurality of parameters. In some implementations, the mood score is further determined using the first value for each of the parameters, the second value for each of the parameters, or a combination of the first and the second valued for each of the parameters.

In some implementations, the mood score is determined as a sum of a first product and a second product. The first product is the product of the second value for a first parameter selected from the plurality of parameters and the associated determined base weight value for the first parameter. The second product is the product of the second value for a second parameter selected from the plurality of parameters and the associated determined base weight value for the second parameter. Equation 1 is a mathematical expression of this function.

MS=W₁P₁(d₂)+W₂P₂(d₂). Equation 1

MS is the mood score. W₁ is the determined base weight for the first parameter P₁, where P₁ is data associated with the second day, d₂. W₂ is the determined base weight for the second parameter P₂, where P₂ is data associated with the second day d₂.

Equation 1 can be expanded to include all of the parameters and can be expressed as the sum of products over all the parameters as shown in Equation 2.

MS=Σₖ₌₁ⁿWₖPₖ(d₂); wherein n is the number of the plurality of parameters. Equation 2

In both equations 1 and 2, the parameter is the value for the second day, d₂, which is after the first day. In some implementations, the parameter can be the value associated with any second time that is after a first time. For example, the second time can be an hour, two hours, three hours, six hours or 12 hours after the first time. The second day can also be any day after the first day. For example, the second day can be a year, six months, three months, a month, 10 days, 5 days, 4 days, 2 days, or 1 day after the first day. Accordingly, in some implementations, the first time period is about 1 to 365 days, about 1 to 182 days, 1 to 90 days, 1 to 30 days, 1 to 5 days, 1 to 4 days, 1 to 2 days or 1 day.

The function to determine the mood score can also be normalized. For example, the mood score can be divided by the maximum sum obtainable by equations 1 and 2 and expressed as a percentage of the maximum mood score, or on any normalized scale. For example, as shown by equation 3.

MS=SΣₖ₌₁ⁿWₖPₖ(d₂)/Σₖ₌₁ⁿWₖₘₐₓPₖₘₐₓ(d₂) Equation 3

The scaling factor, S, can be any value. For example, S is 100 for a percentage, or 10 for a mood scale from 0 to 10. Wₖₘₐₓ is the maximum weight factor for parameter Pₖ. Pₖₘₐₓ is the maximum value for the parameter Pₖ. The maximum value Pₖₘₐₓ can be, for example, the maximum healthy threshold for the parameter, or a value 1 to 10 times the healthy threshold parameter.

Other forms of normalization can be used. For example, the parameter data can be normalized to all be with the same or similar values. In some implementations, the data is normalized to be between about 1 and 100, 1 and 50 or 1 and 10. In some implementations, the data is normalized and the signs of the data are unified so that a positive slope of the plotted data corresponds to a positive trend indicator, and the slopes are about the same in magnitude.

In some implementations, the mood score is determined based on a combination of the first and second value for each of the parameters. For example, the mood score can be determined by the mean or the average of the first and second values for each of the parameters.

In some implementations, the mood score can be determined based on the base weight and a first average value which is an average value calculated using the first and second value for each of the plurality of parameters. For example, the mood score can be a sum of a first determined product and a second determined product. The first determined product is the product of the first average value of a first parameter selected from the plurality of parameters and the associated determined base weight value for the first parameter. The second determined product is the product of the first average value of a second parameter selected from the plurality of parameters and the associated determined base weight value for the second parameter. Equation 4 is a mathematical expression of this function.

MS=W₁P̅₁(d₁,d₂)+W₂P̅₂(d₁,d₂). Equation 4

MS, W₁, and W₂ are as previously defined. P̅₁(d₁, d₂) is an average of the value for the first parameter on the first day, and the value of the first parameter on the second day. P̅₂ (d₁, d₂) is an average of the value of the second parameter on the first day and the value of the second parameter on the second day.

Equation 4 can be expanded to include all of the parameters and can be expressed as the sum of products over all the parameters as shown in Equation 5.

MS=Σₖ₌₁ⁿWₖP̅ₖ(d₁,d₂). Equation 5

In some implementations more values are used to calculate the average Pₖ. For example, values for the parameters on any day between first day d₁ and second day d₂ can be included to calculate the average, such as each day between d₁ and d₂. In some implementations, the mean, median, or range of values is used rather than the average of the parameters.

The function to determine the mood score can also be normalized, such as had been previously described. For example, the mood score can be divided by the maximum sum obtainable by equations 4 and 5 and expressed as a percentage of the maximum mood score, or on any normalized scale. For example, as shown by equation 6.

MS=SΣₖ₌₁ⁿWₖP̅ₖ(d₁,d₂)/Σₖ₌₁ⁿWₖₘₐₓPₖₘₐₓ(d₁,d₂) Equation 6

Blocks 860, 870, 880 and 890 show optional steps for implementation of process 800.

Block 860 includes receiving an intermediate value for each of the plurality of parameters associated with the user on an intermediate day. The intermediate day is any day that is after the first day, and before the second day. For example, the intermediate day can be one day, two days, three days, four days, five days, a week, ten days, a month, three months, 100 days, six months or a year before the second day, provided the intermediate day is after the first day. Accordingly, in some implementations, the first time period is about 1 to 364 days, about 1 to 181 days, 1 to 89 days, 1 to 29 days, 1 to 4 days, 1 to 3 days, 1 to 2 days or 1 day.

In block 870, the user parameters received in steps 820 and step 860 are used to determine an intermediate trend indication for each of the user parameters. The intermediate trend indication is based at least on the intermediate values, the second values, and an intermediate time period. The intermediate time period is the period of time between the intermediate day and the second day. The trend indication can be determined by statistical methods such as line fitting as previously described with reference to FIGS. 5 to 7.

In some implementations, the intermediate trend indication for each of the plurality of parameters is also based at least in part on a range of healthy threshold values for each of the plurality of parameters. Optionally, determining the intermediate trend indication for each of the plurality of parameters includes determining a rate of change between at least the intermediate value and the second value for each of the plurality of parameters during the intermediate time period. For example, according to some aspects, the rate of change is associated with a slope of a line fitted to at least the intermediate value and the second value.

In some implementations, the intermediate trend indication for a first one of the plurality of parameters is a positive trend indication responsive to a determination that the slope of the fitted line is greater than a first slope threshold. For example, wherein the first slope threshold is 0.5, 0.3, 0.2, 0.1, 0.01, or 0.05.

In some implementations, the intermediate trend indication for a first one of the plurality of parameters is a negative intermediate trend indication responsive to a determination that the slope of the fitted line is less than a second slope threshold. For example, wherein the second slope threshold is −0.5, −0.3, −0.2, −0.1, −0.01, or −0.05.

Optionally, the intermediate trend indication for a first one of the plurality of parameters is a stable-good intermediate trend indication responsive to determining that; (i) the slope of the fitted line is within a range of slope values, and (ii) an average of at least the intermediate value and the second value is within a range of healthy threshold values. In some implementations, at least one of the intermediate value and the second value is within the range of healthy threshold values for the intermediate trend indication to be stable-good. In some implementations, the intermediate value and the second value are within the range of the healthy threshold values for the intermediate trend indication to be stable-good.

Optionally, the intermediate trend indication for a first one of the plurality of parameters is a stable-bad trend indication responsive to determining that; (i) the slope of the fitted line is within a range of slope values, and (ii) an average of at least the intermediate value and the second value is outside a range of healthy threshold values. In some implementations, at least one of the intermediate value and the second value is outside of the range of healthy threshold values for the intermediate trend indication to be stable-bad. In some implementations, at least the intermediate value and the second value are outside of the range of healthy threshold values for the intermediate trend indication to be stable-bad.

An intermediate base weight value for each of the plurality of parameters is determined in block 880. The intermediate base weight value is determined based on the intermediate time period and the associated intermediate trend indication. The intermediate-based weigh value can be determined as previously described, e.g., Table 1, FIGS. 5 to 7. For example, in the data presented in Table 1, the short-term time is equivalent to the intermediate time period, and the long-term time period is equivalent to the first time period.

In block 890, the mood score is determined based at least on the base weight value and the intermediate base weight value for each of the plurality of parameters. In some implementations, the mood score is further determined using the intermediate value for each of the parameters, the second value for each of the parameters, or a combination of the first and the second valued for each of the parameters.

In some implementations, the mood score is determined as a sum of products, as illustrated by equation 7.

MS=Wᵢₙₜ₁P₁(dᵢₙₜ)+Wᵢₙₜ₂P₂(dᵢₙₜ). Equation 7

Wᵢₙₜ₁ is the intermediate base weight associated with the first parameter. Wᵢₙₜ₂ in the intermediate base weight is associated with the second parameter. P₁ is the first parameter value, received or measured on the intermediate date dᵢₙₜ. P₂ is the second parameter value, received or measured on dᵢₙₜ.

Equation 7 can be expanded to include all the possible parameters, as shown in equation 8.

MS=Σₖ₌₁ⁿWᵢₙₜₖPₖ(dᵢₙₜ) Equation 8

The mood score can also be normalized, for example, as shown in equation 9.

MS=SΣₖ₌₁ⁿWᵢₙₜₖPₖ(dᵢₙₜ)/Σₖ₌₁ⁿWᵢₙₜₖₘₐₓPₖₘₐₓ(dᵢₙₜ) Equation 9

Wᵢₙₜₖ max is the maximum intermediate weight factor for the corresponding Pₖ.

In some implementations, the mood score is determined based on a combination of the intermediate and second values for each of the parameters. For example, the mood score can be determined by the mean or the average of the intermediate and second values for each of the parameters.

In some implementations, the mood score can be determined based on the intermediate base weight and an intermediate average value. The intermediate average value is calculated using the intermediate and second values for each of the plurality of parameters. For example, the mood score can be a sum of a third determined product and a fourth determined product. The third determined product is the product of the intermediate average value of a first parameter selected from the plurality of parameters and the associated determined intermediate base weight value for the first parameter. The fourth determined product is the product of the intermediate average value of a second parameter selected from the plurality of parameters and the associated determined intermediate base weight value for the second parameter. Equation 10 is a mathematical expression of this function.

MS=Wᵢₙₜ₁P̅₁(dᵢₙₜ,d₂)+Wᵢₙₜ₂P̅₂(dᵢₙₜ,d₂). Equation 10

MS, Wᵢₙₜ₁, and Wᵢₙₜ₂ are as previously defined. P̅₁(dᵢₙₜ, d₂) is an average of the value for the first parameter on the intermediate day, and the value of the first parameter on the second day. P̅₂ (dᵢₙₜ, d₂) is an average of the value of the second parameter on the intermediate day and the value of the second parameter on the second day.

Equation 10 can be expanded to include all of the parameters and can be expressed as the sum of products over all the parameters as shown in Equation 11.

MS=Σₖ₌₁ⁿWₖP̅ₖ(dᵢₙₜ,d₂). Equation 11

In some implementations more values are used to calculate the average P̅ₖ. For example, values for the parameters on any day between intermediate day, dᵢₙₜ, and second day, d₂, can be included to calculate the average, such as each day between dᵢₙₜ and d₂. In some implementations, the mean, median, or range of values is used rather than the average of the parameters.

The function to determine the mood score can also be normalized as previously described. For example, the mood score can be divided by the maximum sum obtainable by equations 10 and 11 and expressed as a percentage of the maximum mood score, or on any normalized scale. For example, as shown by equation 12.

MS=SΣₖ₌₁ⁿWᵢₙₜₖP̅ₖ(dᵢₙₜ,d₂)/Σₖ₌₁ⁿWᵢₙₜₖₘₐₓPₖₘₐₓ(dᵢₙₜ,d₂). Equation 12

In some implementations, the mood score is a function of the base weight, the intermediate base weight, and the parameter values for the first day, d₁, the intermediate day, dᵢₙₜ, and the second day, d₂. Examples of some possible functions are listed in Table 2, which are combinations of the previously described functions. Other functions are possible and contemplated. For example, second-, third-, and fourth-order functions.

TABLE 2
Some Mood Score functions

| Equa- | |
|---|---|
| tion | |
| # | Equation: |
| 12 | MS = W₁P₁(d₂) + W₂P₂(d₂) + Wᵢₙₜ₁P₁(dᵢₙₜ) + Wᵢₙₜ₂P₂(dᵢₙₜ) |
| 13 | MS = Σₖ₌₁ⁿ{Wₖ Pₖ (d₂) + Wᵢₙₜₖ Pₖ (dᵢₙₜ)} |
| 14 | MS = W₁P̅₁(d₁, d₂) + W₂P̅₂(d₁, d₂) + Wᵢₙₜ₁P̅₁(dᵢₙₜ, d₂) + |
| | Wᵢₙₜ₂P̅₂(dᵢₙₜ, d₂). |
| 15 | MS = Σₖ₌₁ⁿ{Wₖ P̅ₖ (d₁, d₂) + Wₖ P̅ₖ (dᵢₙₜ, d₂)}. |

The mood score values can be normalized, for example, as previously described, by including a scaling factor and dividing by the maximum possible mood score values.

In some implementations, the determined base weight value for each of the plurality of parameters is based on a set of predetermined base weight values. Alternatively or additionally, according to some implementations, the determined intermediate base weight value for each of the plurality of parameters is based on a set of predetermined initial intermediate base weight values. A, a true mood score is received, wherein the true mood score is associated with the user and the second day that is subsequent to the first day, or subsequent to the intermediate day. In some cases, the true mood score is used to modify the predetermined initial base weight values, the predetermined initial intermediate base weight values, or both.

The true mood score is a mood score associated with the user and a specific day, such as the second or current day. The true mood score can be determined by, for example, consultation with one or more clinicians, care providers, medical professionals, or mental health professionals. For example, the user can meet with a mental health professional, such as a psychiatrist, who can pose questions and determine the person's mood or mood disorder, such as depression. The true mood score can be scaled similar to the determined mood score, for example, to provide easy comparison. A numerical value can be assigned based on the mental health professional's observation. For example, as listed in Table 3, which is a mood scale accessed on the world wide web Sep. 15, 2020 at https://blueprintzine.com/2017/10/08/writing-mood-scales-a-guide/ and is incorporated here by reference.

TABLE 3
True Mood Score

| | Mood | |
|---|---|---|
| Condition | score | Observation |
| Mania | 10 | Total loss of judgment, exorbitant spending, religious delusions and |
| | | hallucinations |
| | 9 | Lost touch with reality, incoherent, no sleep, paranoid and vindictive, |
| | | reckless behavior |
| Hypomania | 8 | Inflated self-esteem, rapid thoughts and speech, counter-productive |
| | | simultaneous tasks. |
| | 7 | Very productive, everything to excess (phone calls, writing, smoking, coffee), |
| | | charming and talkative |
| Balanced | 6 | Self-esteem good, optimistic, sociable and articulate, good decisions and |
| | | gets work done |
| | 5 | Mood in balance, no symptoms of depression or mania. Life is going well and |
| | | the outlook is good. |
| | 4 | Slightly withdrawn from social situations, concentration less than usual, |
| | | slight agitation |
| Mild to | 3 | Feelings of panic and anxiety, concentration difficulty and memory poor, |
| moderate | | some comfort in routine |
| depression | 2 | Slow thinking, no appetite, need to be alone, sleep excessive or difficult, |
| | | everything a struggle |
| Severe | 1 | Feelings of hopelessness and guilt, thoughts of suicide, little movement, |
| depression | | impossible to do anything right |
| | 0 | Endless suicidal thoughts, no way out, no movement, everything is bleak and |
| | | it will always be like this. |

The true mood score can be used to test the accuracy of an equation or function that is applied for determining the mood score. The equations can be adjusted accordingly to minimize the error, delta, or residuals between the calculated mood scores and true mood scores. For example, the initial base weights and the initial intermediate base weights can be adjusted so that the determined mood scores more closely match the true mood scores.

In some implementations, the true mood score is used to train an algorithm for predicting the mood score. For example, inputs of the various parameters and base weights described herein can be used for a machine learning algorithm where the true mood score is used for training the algorithm. The machine learning algorithm can use parameters from multiple users over multiple time periods to arrive and an increasingly accurate prediction. In some implementations, the machine learning algorithm can be stored on the memory device 114 and executed by the processor 112 of the control system 110.

According to some aspects, a mitigating action is taken responsive to the mood score. Optionally the mitigation action includes an alert sent to a care provider. In some implementations, the mitigating action is an assignment of additional time with a care provider who can monitor and interact with the user. The mitigation action can include scheduling events for the individual, including therapy or activities. The mitigating action can also include a diagnosis of a mood disorder or psychological disorder, such as ADHD, anxiety, social phobia, major depressive disorder, bipolar disorder, seasonal affective disorder, cyclothymic disorder, premenstrual dysphoric disorder, persistent depressive disorder (or dysthymia), disruptive mood dysregulation disorder, depression related to medical illness, depression induced by substance use or medication, or any combination thereof. The mitigating action can also include prescription of appropriate medications, such as antidepressants, stimulants, or mood-stabilizing medicines. The mitigating action can also include other types of treatment, such as psychotherapy, family therapy, or other therapies. In some implementations, the mitigating action can be an assignment of a therapy animal, such as a dog or cat, to the individual. In some implementations, the mitigation action includes providing soothing or the user's favorite music, a movie, or a story.

### Examples

Overview

To detect depression, a series of inputs from Electronic Health Record (EHR), a Certified Nursing Assistant (CNA), and sensors can be used to determine an output score that is much more frequent than the control variables used to test accuracy. The output score can be a rolling number that can be compared against a control set of data in a Patient Health Questionnaire (e.g., PHQ9). Control data is collected (in many cases by Social Workers) on admission, discharge, and annual assessments. Using the output score that is validated by the control variables, interventions can be automatically generated and tailored to the resident for maximum positive outcomes. In some implementations, the interventions include the treatment (e.g., automatic prescription), or recommendation for treatment (e.g., recommendation to the patient or a care provider), using medications (e.g., antidepressants, stimulants, mood-stabilizing medicines), psychotherapy, family therapy, other therapies, or any combination thereof. In some such implementations, the output score can be displayed on a display device (such as the display device 172 as disclosed herein) for ease of understanding of the treatment and/or recommendation of treatment. In some such implementations, the interventions include automatic treatment of the patient, such as automatic administration of the prescribed medications. After interventions, the process of analyzing data and generating a score (validated by the slower control data) can be repeated. In some implementations, the new score is displayed on a display device (such as the display device 172 as disclosed herein) for monitoring progression of the patient and/or adjustment of the treatment or recommendation. More interventions can be recommended as needed.

Value Proposition

Current data for detecting depression is not collected by members of the facility. Rather, external parties like social workers or group therapy sessions, collect the data. Automating this collection of data by the disclosed methods will allow facility staff to identify and remediate depression faster and more effectively. In addition, the described system will increase the accuracy of diagnosis and/or treatment of the patient.

Samples EHR and Sensor Input Data

Input will be pulled from a caregiver such as MatrixCare's EHRs and any devices or sensors available. Table 4 lists sample EHR Data input, and Table 5 lists sample sensor data.

TABLE 4
EHR Sample Data

| Column Name | Data Tables | Description |
|---|---|---|
| Name | | |
| Date of Birth | | |
| Location | | |
| Month/Season | | |
| PatientID | Facesheet | |
| EUID | Resident Profile | Globally unique identifier across all |
| | | MatrixCare systems. |
| Sex | Facesheet | Gender (M, F, U) |
| RaceCode | Facesheet | Race of the Patient (Asian, Black, |
| | | Hispanic, Native, Unknown, White) |
| MaritalStatusCode | Facesheet | Marital Status of the Patient during |
| | | the event (D, M, S, U, W, X) |
| NoOfFalls | MX SNF RCM O/E | Number of previous falls the patient |
| | | has had so far. |
| Diagnosis | MX Facesheet | Diagnosis during the event (They |
| | | are in the form of ICD 10 codes, |
| | | multiple diagnoses are specified |
| | | separated by comma) |
| DrugList | MX EMAR and MX SNF | List of ‘;’ separated Drugs |
| | Orders | |
| Balance Toilet Unsteady | MX POC | Indicator resident was unsteady |
| Stabilize w/Assist cnt | | using the toilet and needed |
| | | assistance. |
| | | Number of times a service was |
| | | provided for the 15-day time period |
| | | (inclusive of event occurred/not |
| | | occurred date), if the service was |
| | | provided “n” times in a day, it will be |
| | | counted as 1 else it is 0. Values |
| | | range from 0-15 |
| Balance Toilet Unsteady | MX POC | Indicator resident was unsteady |
| Stabilize w/o Assist ent | | using the toilet and did not need |
| | | assistance. |
| | | Number of times a service was |
| | | provided for the 15-day time period |
| | | (inclusive of event occurred/not |
| | | occurred date), if the service was |
| | | provided “n” times in a day, it will be |
| | | counted as 1 else it is 0. Values |
| | | range from 0-15 |
| J0100A | MDS | Received scheduled pain medication |
| | | regimen |
| | | 0. No . . . |
| | | 1. Yes. |
| J0100B | MDS | Received PRN pain medications OR |
| | | was offered and declined? |
| | | 0. No . . . |
| | | 1. Yes. |
| J0100C | MDS | Received non-medication |
| | | intervention for pain |
| | | 0. No . . . |
| | | 1. Yes. |
| Temperature | Medical-Related Data | Temperature readings |
| | (Vitals and Pain) | e.g. 3/12-98.6; 3/15-98.5, 3/17 |
| | | 98.7, 3/20-101.5 (current date) |
| Temperature Deviation | Medical-Related Data | Baseline Value: Last 3 Temperature |
| | (Vitals and Pain) | readings from date recorded |
| | | (excluding the latest value), |
| | | averaged. |
| | | Calculation: a percentage value is |
| | | calculated. Average value subtracted |
| | | from latest temperature and |
| | | converted to a |
| | | percentage. Value can be both |
| | | positive and negative. |
| | | e.g. 3/12-98.6; 3/15-98.5, 3/17 |
| | | 98.7, 3/20-101.5 (current date) |
| | | Average Value of Last 3 readings |
| | | from current date = 98.6 + 98.5 + |
| | | 98.7/3 = 98.6 |
| | | convert (int, 100 * (Current Date |
| | | Value − Avg Value)/Avg Value) = |
| | | ((101.5-98.6)/98.6)* 100 = (2.9/98.6) |
| | | *100 = 2.9 |
| Pulse | Medical-Related Data | Pulse readings from date recorded |
| | (Vitals and Pain) | (excluding the latest value), averaged |
| | | e.g. 3/12-65. 3/15-67, 3/17-65, |
| | | 3/20-67 (current date) |
| Pulse Deviation | Medical-Related Data | Baseline: Last 3 Pulse readings from |
| | (Vitals and Pain) | date recorded (excluding the latest |
| | | value), averaged |
| | | Calculation: a percentage value is |
| | | calculated. Average value subtracted |
| | | from latest Pulse and converted to a |
| | | percentage. Value can be both |
| | | positive and negative. |
| | | e.g. 3/12-65. 3/15-67, 3/17-65, |
| | | 3/20-67 (current date) |
| | | Average Value of Last 3 readings |
| | | from current date = 65 + 67 + 65)/3 = |
| | | 65.6 |
| | | convert (int, 100 * (Current Date |
| | | Value − Avg Value)/Avg Value) = |
| | | ((67-65.6)/65.6)*100 = (1.4/65.6) |
| | | *100 = 2.13 |
| Respiration | Medical-Related Data | Respiration readings from date |
| | (Vitals and Pain) | recorded (excluding the latest value), |
| | | averaged. |
| | | e.g. 3/12-23; 3/15-20, 3/17-20, |
| | | 3/20-20 (current date) |
| Respiration Deviation | Medical-Related Data | Baseline: Last 3 Respiration readings |
| | (Vitals and Pain) | from date recorded (excluding the |
| | | latest value), averaged. |
| | | Calculation: a percentage value is |
| | | calculated. Average value subtracted |
| | | from latest Respiration and |
| | | converted to a |
| | | percentage. Value can be both |
| | | positive and negative. |
| | | e.g. 3/12-23; 3/15-20, 3/17-20, |
| | | 3/20-20 (current date) |
| | | Average Value of Last 3 readings |
| | | from current date = (23 + 20 + 20)/3 = 21 |
| | | convert (int, 100 * (Current Date |
| | | Value − Avg Value)/Avg Value) = |
| | | ((20-21)/21)*100 = (−1/21)* 100 = −4.7 |
| Weight | Medical-Related Data | Weight is measured in Pounds |
| | (Vitals and Pain) | |
| Height | Medical-Related Data | Height is measured in Inches |
| | (Vitals and Pain) | |
| Evidence of Pain | MX POC | Binary Response 1 or 0, “Does |
| | | Resident show evidence of pain (or |
| | | possible pain)? = Yes |

In Table 4, MDS refers to Minimal Data Set, MX refers to the name of the caregiver, MatrixCare, SNF refers to Skilled Nursing Facility, RCM refers to Revenue Cycle Management System, O/E refers to Order Entry, and EMAR refers to Electronic Medication Administration Record.

TABLE 5
Engagement and Sensor Data

| Category | Name | Description |
|---|---|---|
| Sensor | Steps Taken | Number of steps in given period (e.g., 1 day) using a |
| | | personal sensor worn on the wrist or a mobile phone |
| | | equipped with step sensing technology. |
| Sensor | Heart Rate | Average beats per minute for a period (e.g., 1 day) using a |
| | | personal sensor worn on the wrist. |
| Chat/Mic | Spoken language | percentage of spoken words in a measurement period |
| | | (e.g., 1 day) in language other than English |
| | | (or main/primary language of resident) |
| Chat/Mic | Content of Language | percentage of spoken words that are indicative of frustration |
| | | (e.g., swearwords, etc.) in a measurement period (e.g., 1 day) |
| Chat/Mic | Speed of Talking | number of words per minute |
| Video | Facial Expression | number of times certain facial expressions are detected |
| chatbot | | during the period (e.g., 1 day/1 chatbot session) (the facial |
| | | expressions of interest include, e.g., frown, frustrated, etc.) |

Machine Learning Process

Data from Tables 4 and 5 can be analyzed via machine learning algorithms to predict outcomes quickly and with the same or better quality as the existing methods of detecting common mood patterns. The raw collected data 5 is processed to organize and “clean” the data. This includes generating input datasets and removing any known errors that will skew results. The data will also be normalized for analysis purposes (see below). When data is cleaned and ready for use, a variety of models can be tested during the training phase, and a score for the model can be compared against a standard clinical data set or a true mood score. The true mood score can be determined, for example, by the questionnaire shown in Table 3, or a PHQ-9 questionnaire as shown in Table 7 below).

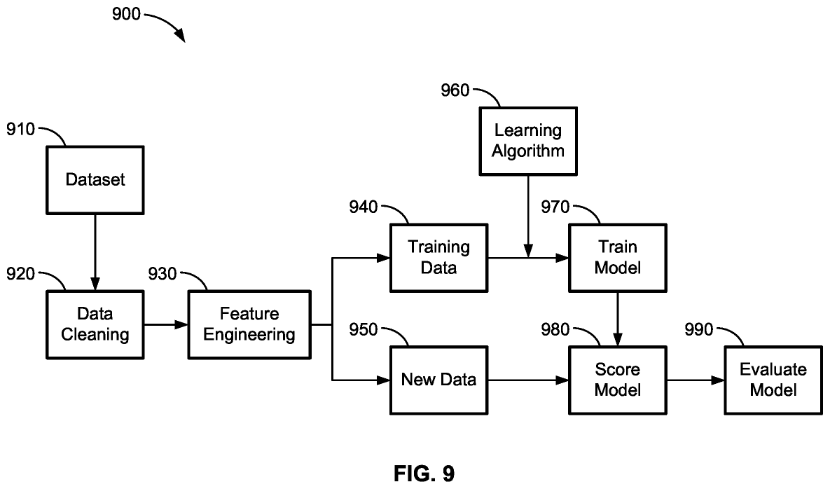

The steps for a machine learning algorithm 900 are shown with reference to FIG. 9. The initial step is collecting the data 910. This is as described above and includes populating Tables 4 and 5. The data set is cleaned at step 920, also as described above.

Feature engineering 930 is then applied to the data. Feature engineering is the process of using domain knowledge of the data to create features that make machine learning algorithms work. For example, combining features such as steps taken and the age of a subject might make the algorithm work better. Feature engineering can also include removing features that are judged to be unimportant.

Training data 940 is then input into a learning algorithm 960 to train the model 970. These steps relate to determining values for weights and bias for the model. Examples for which the output is known are used for training.

Any useful learning algorithm 960 can be used, and broadly are selected from; Supervised learning, Unsupervised learning, and Reinforcement learning. Supervised learning algorithms consist of a target/outcome variable (or dependent variable) which is to be predicted from a given set of predictors (independent variables). Using these sets of variables, a function is generated that maps inputs to desired outputs. The training process continues until the model achieves a desired level of accuracy with the training data. Examples of Supervised Learning include; Regression, Decision Tree, Random Forest, KNN, and Logistic Regression. Unsupervised learning algorithms, are used when there is no target or outcome variable to predict/estimate. This can be used for clustering populations into different groups, which is widely used for segmenting customers into different groups for specific intervention. Examples of Unsupervised Learning include: Apriori algorithm, and K-means. With Reinforcement Learning algorithms, the machine is trained to make specific decisions. The machine is exposed to an environment where it trains itself continually using trial and error. This machine-learning algorithm learns from past experience and tries to capture the best possible knowledge to make accurate decisions. An example of Reinforcement Learning is the Markov Decision Process.

New data 950 can then be input into the initially trained model, and the model is scored at step 980 based on how well it correctly predicts the output. For example, in this case, the output is a mood score. The model can then be modified by more iterations of training the model. The model is then evaluated at step 990.

### Examples of Data Normalization

A major challenge is presenting the following data in a common scale for analysis. For example, Balance Toilet Unsteady Stabilize w/ Assist cnt—This is a binary answer of 1 or 0 each day and then summed into a rolling past 15-day value. This is of limited value and can be improved by into a 3-part value:

0=Unknown

1=Observed, Resident did not need assistance; and

2—Observed, Resident needed assistance.

As another example, steps taken is sensor data that is recorded almost continuously, but measurements are taken in daily increments. To convert this to a common scale, a baseline is set and delta from that baselines is tracked. A percentage from the baseline can be used for analysis.

As yet another example, a subject's weight is measured each day but does not fall neatly on a scale from 1 to 10. With supplemental information, this may be converted to a body mass index (BMI). The BMI can be more easily portioned into a meaningful scale to input into the learning algorithm to determine a mood score.

Algorithm

The algorithm provides a neutral adjusted scale from 0 to 10, where 5 is feeling normal, greater than 5 is better than normal, and less than 5 is feeling worse than normal. Table 6 illustrates algorithm-related data such as input types, initial weights, and accuracy.

TABLE 6

| | | | Initial | | |
|---|---|---|---|---|---|
| Type | Source | Data Points | Weight | Accuracy | Scale |
| Input | Sensor Data | Objective data from sensors | 4 | 80% | 0 to 10 |
| | | or calculates. | | | |
| Input | Point of Care | Mostly objective data from | 2 | 60% | 0 to 10 |
| | | informal assessments. | | | |
| | | MatrixCare secret source | | | |
| | | questioner. | | | |
| Input | Progress Notes | Subjective data that needs to | 1 | 20% | 0 to 10 |
| | | be parsed. | | | |
| Output | (new algorithm) | | | | |
| Control | PHQ-9 | Control data to compare | 5 | 80% | 0 to 10 |
| | | against for accuracy. | | | |
| Control | MDS | Mostly objective data from | 4 | 80% | 0 to 10 |
| | | formal assessments. | | | |
| | | Government regulated. | | | |

Sample Data Analysis

Time periods of data evaluation:

(a) Volatile Trends:
i. These are intraday measurements. These are measured in very short periods of time, like seconds or minutes. This data helps to feed predictive trends but is difficult to interpret on its own and thus is more useful with supporting data.

(b) Predictive Trends
i. These are daily measurements. Multiple days of data can typically create very useful data, and mixed with supporting data can lead to interventions that increase the chance of positive outcomes.

(c) Baseline Trends
i. These are monthly measurements. Longer periods of trends that tend to align with traditional ways of measurement like the PDQ-9.

(d) Long-term Trends
i. This category spans anything longer than a month.

(e) Trendline modifiers
i. Volatile Trend=measurement*10%
ii. Predictive Trend=measurement*50%
iii. Baseline Trend=measurement*100%
iv. Long-term Trend=measurement*100%

Calculations over time will create a person-specific new norm of the baseline. This means that if a subject trends lower than average for a long period of time, that subject's trend becomes the subject's individualized “new norm.” The algorithm will detect changes in the new norms.

FIGS. 10-13 are plots of collected data.

FIG. 10 depicts sensor data that is sampled daily. The sensor data can be, for example, a motion sensor that detects the steps taken. Sensor data has a high frequency (daily) with a high accuracy rating.

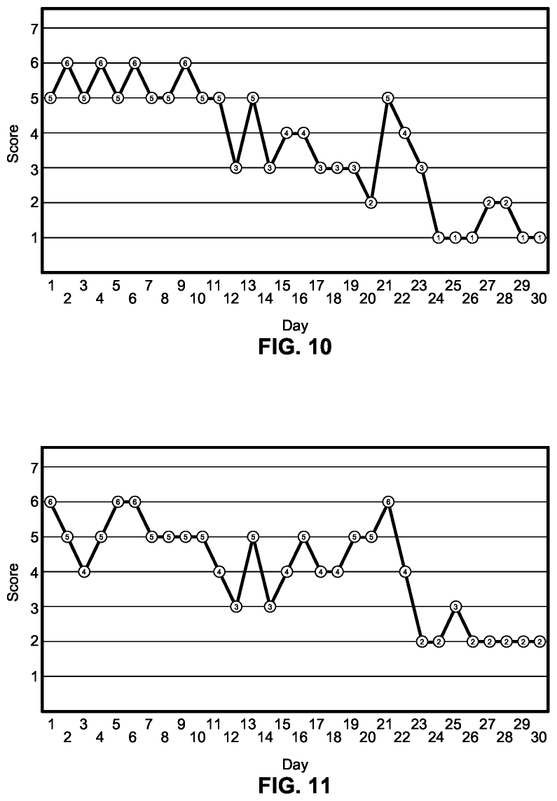

FIG. 11 depicts POC data. POC data is from the EHR system and has a high frequency as well (daily). The data is more subjective than sensor data since it requires some judgment from a POC provider such as a nurse.

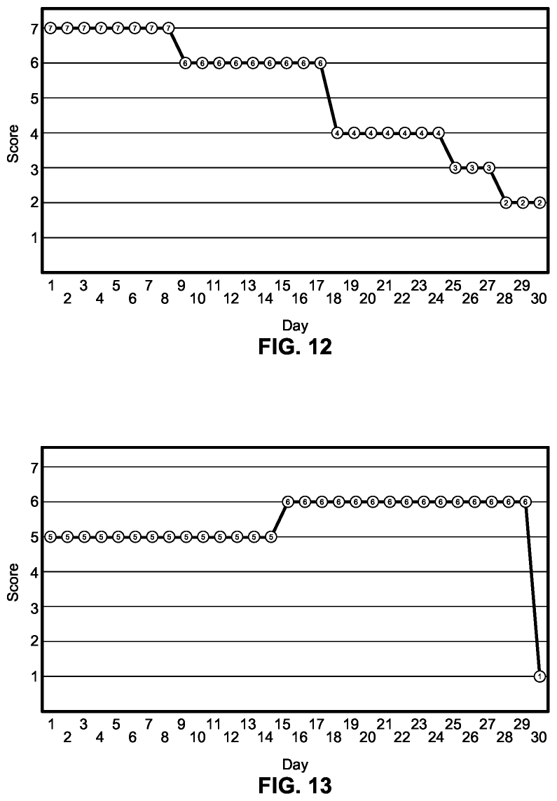

FIG. 12 depicts Progress Notes data. Progress Notes data is highly subjective data. These data have a high potential for refinement by mining the free text algorithms. This data is hindered by lower frequency (a few times a month) and non-mandated intervals. The infrequency of the sampling is indicated by the repetition of values over several days in FIG. 12.

FIG. 13 depicts Control Data. Control data is the gold standard data and is obtained from Minimal Data Set (MDS), the True Mood Score, and items like the PHQ-9, which are accepted by the industry as high quality but have a very low frequency. MDS evaluations can be months apart and PHQ-9 and Mood Score varies by facility. The low sampling frequency is depicted by the repeat values over several days.

Control Data

The PHQ-9 can be used as control data to determine if the above process is working as expected. The PHQ-9 is administered at periodic intervals so is much less frequent than what an automatic algorithm uses.

TABLE 7
PHQ-9 Data

| | | | | More than | Nearly |
|---|---|---|---|---|---|
| | | Not | Several | half the | Every |
| Item | Description | at all | Days | days | day |
| 1 | Little interest or pleasure in doing | 0 | 1 | 2 | 3 |
| | things | | | | |
| 2 | Feeling down, depressed, or hopeless | 0 | 1 | 2 | 3 |
| 3 | Trouble falling or staying asleep, or | 0 | 1 | 2 | 3 |
| | sleeping too much | | | | |
| 4 | Feeling tired or having little energy | 0 | 1 | 2 | 3 |
| 5 | Poor appetite or overeating | 0 | 1 | 2 | 3 |
| 6 | Feeling bad about yourself-or that | 0 | 1 | 2 | 3 |
| | you are a failure or have let yourself or | | | | |
| | your family down | | | | |
| 7 | Trouble concentrating on things, such | 0 | 1 | 2 | 3 |
| | as reading the newspaper or watching | | | | |
| | television | | | | |
| 8 | Moving or speaking so slowly that | 0 | 1 | 2 | 3 |
| | other people could have noticed. Or | | | | |
| | the opposite-being so fidgety or | | | | |
| | restless that you have been moving | | | | |
| | around a lot more than usual | | | | |
| 9 | Thoughts that you would be better off | 0 | 1 | 2 | 3 |
| | dead, or of hurting yourself in some way | | | | |

| | | | |
|---|---|---|---|
| Add columns: | 2 | 10 | 3 |

| | |
|---|---|
| TOTAL: | 15 |

| | | | |
|---|---|---|---|
| 10 | If you checked off any problems, how | Not difficult at all | |
| | difficult have these problems made it for | Somewhat difficult | X |
| | you to do your work, take care of things at | Very difficult | |
| | home, or get along with other people | Extremely difficult | |

In some implementations, the mood score is determined using a combination of sensor data and EHR data. For example, in some implementations, the steps taken by the subject, the heart rate of the subject, the Balance Toilet Unsteady Stabilized with Assistance count, and POC HER-evidence of pain are used to determine the mood score.

One or more elements or aspects or steps, or any portion(s) thereof, from one or more of any of claims 1 to 88 below can be combined with one or more elements or aspects or steps, or any portion(s) thereof, from one or more of any of the other claims 1 to 88 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving a first verbal communication value for each of a plurality of parameters from an audio sensor, each of the first values being associated with (i) a user and (ii) a first day;
receiving a second value for each of the plurality of parameters from a mobile device or a wearable device, each of the second values being associated with (i) the user and (ii) a second day that is subsequent to the first day;
determining, for each of the plurality of parameters, a trend indication, the trend indication for each of the plurality of parameters being based at least in part on the first values, the second values, and a first time period via a processor;
determining a base weight value for each of the plurality of parameters, the base weight value for each of the plurality of parameters being based at least in part on the first time period and the associated determined trend indication; and
determining a mood score, based on the base weight value for each of the plurality of parameters.

2-8. (canceled)

9. The method of claim 1, wherein determining the trend indication for each of the plurality of parameters includes determining a rate of change between at least the first value and the second value for each of the plurality of parameters during the first time period, and wherein the rate of change is associated with a slope of a line that is fitted to at least the first value and the second value.

10. The method of claim 9, wherein the trend indication for a first one of the plurality of parameters is one of a positive trend indication responsive to a determination that the slope of the fitted line is greater than a first slope threshold or a negative trend indication responsive to a determination that the slope of the fitted line is less than a second slope threshold.

11-13. (canceled)

14. A method comprising:
receiving a first value for each of a plurality of parameters, each of the first values being associated with (i) a user and (ii) a first day;
receiving a second value for each of the plurality of, each of the second values being associated with (i) the user and (ii) a second day that is subsequent to the first day;
determining, for each of the plurality of parameters, a trend indication, the trend indication for each of the plurality of parameters being based at least in part on the first values, the second values, and a first time period, and determining a rate of change between at least the first value and the second value for each of the plurality of parameters during the first time period, wherein the rate of change is associated with a slope of a line that is fitted to at least the first value and the second value;
determining a base weight value for each of the plurality of parameters, the base weight value for each of the plurality of parameters being based at least in part on the first time period and the associated determined trend indication; and
determining a mood score, based on the base weight value for each of the plurality of parameters,
wherein the trend indication for a first one of the plurality of parameters is either a stable-good trend indication responsive to determining that:
(i) the slope of the fitted line is within a range of slope values; and
(ii) an average of at least the first value and the second value is within a range of healthy threshold values; or
a stable-bad trend indication responsive to determining that:
(i) the slope of the fitted line is within a range of slope values; and
(ii) an average of at least the first value and the second value is outside a range of healthy threshold values.

15-20. (canceled)

21. The method of claim 1, wherein the determined base weight value for each of the plurality of parameters is based on a set of predetermined initial base weight values.

22. The method of claim 21, further comprising:
receiving a true mood score, the true mood score being associated with:
(i) the user; and
(ii) the second day that is subsequent to the first day; and
modifying the set of predetermined initial base weight values based at least in part on the true mood score.

23. (canceled)

24. The method of claim 22, wherein the modifying the set of predetermined initial base weight values includes using a machine learning algorithm.

25. The method of claim 1, further comprising:
receiving an intermediate value for each of the plurality of parameters, each one of the intermediate values being associated with:
(i) the user, and
(ii) an intermediate day that is subsequent to the first day and prior to the second day;
wherein the trend indication is also based on the intermediate values.

26. The method of claim 25, further comprising;
determining, for each of the plurality of parameters, an intermediate trend indication, the intermediate trend indication for each of the plurality of parameters being based at least in part on the intermediate values, the second values, and an intermediate time period; and
determining an intermediate base weight value for each of the plurality of parameters, the intermediate base weight value for each of the plurality of parameters being based at least in part on the intermediate time period and the associated determined intermediate trend indication,
wherein determining the mood score is further based on the intermediate base weight value for each of the plurality of parameters.

27-32. (canceled)

33. The method of claim 26, wherein the determining the intermediate trend indication for each of the plurality of parameters includes determining a rate of change between at least the intermediate value and the second value for each of the plurality of parameters during the intermediate time period.

34. The method of claim 33, wherein the rate of change is associated with a slope of a line fitted to at least the intermediate value and the second value.

35. The method of claim 34, wherein the intermediate trend indication for a first one of the plurality of parameters is one of a positive trend indication responsive to a determination that the slope of the fitted line is greater than a first slope threshold; or a negative trend indication responsive to a determination that the slope of the fitted line is less than a second slope threshold.

36-38. (canceled)

39. The method of claim 34, wherein the intermediate trend indication for a first one of the plurality of parameters is one of a stable-good intermediate trend indication responsive to determining that (i) the slope of the fitted line is within a range of slope thresholds and (ii) an average of at least the intermediate value and the second value are a range of healthy threshold values; or a stable-bad trend indication responsive to determining that (i) the slope of the fitted line is within a range of slope thresholds and (ii) an average of at least the intermediate value and the second value are outside the range of healthy threshold values.

40-50. (canceled)

51. The method of claim 1, wherein each of the plurality of parameters is determined based at least in part on data generated by one or more sensors, wherein the one or more sensors is a microphone, a camera, a pressure sensor, a temperature sensor, or a motion sensor.

52. The method of claim 1, wherein each of the plurality of parameters is determined based at least in part on data generated by one or more sensors, wherein at least one sensor is physically coupled to or integrated with a user device; physically coupled to an activity tracker; or physically coupled to a heartrate monitor.

53-54. (canceled)

55. The method of claim 1, wherein the plurality of parameters includes a spoken language during verbal communication, content of language during verbal communication, speed of talking during verbal communication, length of pauses between sentences during verbal communication, mean pitch during verbal communication, peak pitch during verbal communication, mean volume during verbal communication, peak volume during verbal communication, minimal volume during verbal communication, force on keyboard during typed communication, speed of typing during typed communication, length of pauses between entries during typed communication, frequency of communication during typed communication, frequency of communication during verbal communication, confidence of user speech during verbal communication, breathing rate information, heart rate information, temperature information, physical activity information, blood pressure information, activity information, quality of sleep information, social media interaction information, mood information, interest or pleasure in activities, facial expression information, tiredness and overall energy, or any combination thereof.

56. The method of claim 55, wherein the spoken language during verbal communication includes at least one of the percentage of non-primary language spoken by the user, or the number of swear or frustration words spoken by the user.

57-64. (canceled)

65. The method of claim 1, further comprising determining a mental state of the user based at least in part on the mood score, wherein the mental state is determined to be a first mental state responsive to the mood score satisfying a first range of values, and the mental state is determined to be second mental state responsive to the mood score satisfying a second range of values.

66-67. (canceled)

68. The method of claim 65, wherein the mental state is selected from one or more of mania, happiness, euthymia or a neutral mood, sadness, depression, anxiety, apathy, and irritability, and wherein the method further comprising treating the user for mania, depression, anxiety, apathy, or irritability.

69-82. (canceled)

83. A system for diagnosing a user based on a mood score, the system comprising:
one or more sensors configured to generate a plurality of parameters associated with the user;
a memory storing machine-readable instructions; and
a control system including one or more processors configured to execute the machine-readable instructions to:
receive a first value for each of the plurality of parameters, each of the first values being associated with (i) the user and (ii) a first day;
receive a second value for each of the plurality of parameters, each of the second values being associated with (i) the user and (ii) a second day that is subsequent to the first day;
determine, for each of the plurality of parameters, a trend indication, the trend indication for each of the plurality of parameters being based at least in part on the first values, the second values, and a first time period;
determine a base weight value for each of the plurality of parameters, the base weight value for each of the plurality of parameters being based at least in part on the first time period and the associated determined trend indication; and
determine the mood score, based on the base weight value for each of the plurality of parameters.

84-88. (canceled)
